(19) United States
(12) Patent Application Publication
(10) Pub. No.: US 2006/0198875 A1
(43) Pub. Date: Sep. 7, 2006

(54) Core assembly for essential oil

(76) Inventor: Ming-Shao Lu, Jia Yi Hsien (TW)

Correspondence Address: NIKOLAI & MERSEREAU, P.A., 900 SECOND AVENUE SOUTH, SUITE 820, MINNEAPOLIS, MN, 55402, US

(21) Appl. No.: 11/069,508

(22) Filed: Mar. 1, 2005

(51) Int. Cl. A61K 9/70; A61K 36/18

(52) U.S. Cl. 424/443; 424/725

(57) ABSTRACT

A core assembly for essential oil includes an oil suction member, and a core head mounted on and connected to a top end of the oil suction member. Thus, the core head is combined with the oil suction member, so that the essential oil absorbed by the capillary effect the oil suction member is distributed on the core head evenly and smoothly so as to contact with the air completely, thereby enhancing the volatile effect of the essential oil so as to provide a fragrant smell.

### BACKGROUND OF THE INVENTION

1. Field of the Invention

The present invention relates to a core assembly, and more particularly to a core assembly for essential oil.

2. Description of the Related Art

A conventional core 50 in accordance with the prior art shown in FIG. 9 is mounted on the top of a container 52 containing essential oil therein. Thus, the core 50 is heated to spread the essential oil contained in the container 52 outward so as to provide a fragrant smell. However, the essential oil is easily burnt, thereby easily causing danger to a user when the essential oil is upset unintentionally.

Another conventional core includes an oil absorbing member and a potter's clay combined with the oil absorbing member. Thus, the potter's clay has a porous structure, so that the essential oil absorbed by the oil absorbing member is easily volatile by the capillary action of the potter's clay to provide a fragrant smell without having to heat the core, thereby preventing the core from causing danger to a user due to the heating action. However, the potter's clay cannot absorb the essential oil from the oil absorbing member efficiently, so that the capillary action of the potter's clay is limited, thereby decreasing the volatile effect of the essential oil.

### SUMMARY OF THE INVENTION

In accordance with the present invention, there is provided a core assembly, comprising an oil suction member, and a core head mounted on and connected to a top end of the oil suction member.

The primary objective of the present invention is to provide a core assembly for essential oil, wherein the core head is combined with the oil suction member, so that the essential oil absorbed by the capillary effect the oil suction member is distributed on the core head evenly and smoothly so as to contact with the air completely, thereby enhancing the volatile effect of the essential oil so as to provide a fragrant smell.

Another objective of the present invention is to provide a core assembly for essential oil, wherein the essential oil is volatilized naturally without having to heat the core assembly, so that the core assembly is used safely, thereby preventing the core assembly from causing danger to a user due to a heating action.

A further objective of the present invention is to provide a core assembly for essential oil, wherein the core head is made of paper material, thereby decreasing costs of fabrication.

A further objective of the present invention is to provide a core assembly for essential oil, wherein the core head has different patterns, thereby enhancing the aesthetic quality of the core assembly.

A further objective of the present invention is to provide a core assembly for essential oil, wherein after the oil suction member absorbs saturated essential oil, a contraction film is coated around a periphery of the oil suction member to closely seal the oil suction member, so that the core assembly is portable to facilitate storage and transportation of the core assembly.

Further benefits and advantages of the present invention will become apparent after a careful reading of the detailed description with appropriate reference to the accompanying drawings.

### BRIEF DESCRIPTION OF THE DRAWINGS

FIG. 1 is a perspective view of a core assembly for essential oil in accordance with the preferred embodiment of the present invention;

FIG. 2 is an exploded perspective view of the core assembly as shown in FIG. 1;

FIG. 3 is a perspective view showing the core assembly being mounted on a container;

FIG. 4 is a perspective view of a core assembly for essential oil in accordance with another preferred embodiment of the present invention;

FIG. 5 is a perspective view of a core assembly for essential oil in accordance with another preferred embodiment of the present invention;

FIG. 6 is a perspective view of a core assembly for essential oil in accordance with another preferred embodiment of the present invention; FIG. 7 is a perspective view of a core assembly for essential oil in accordance with another preferred embodiment of the present invention;

FIG. 8 is a perspective view of a core assembly for essential oil in accordance with another preferred embodiment of the present invention; and

FIG. 9 is a perspective view of a conventional core assembly for essential oil in accordance with the prior art.

### DETAILED DESCRIPTION OF THE INVENTION

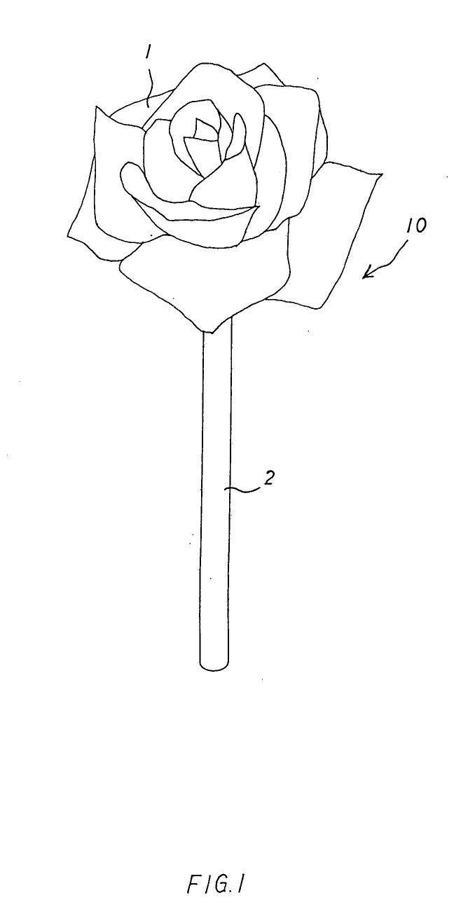

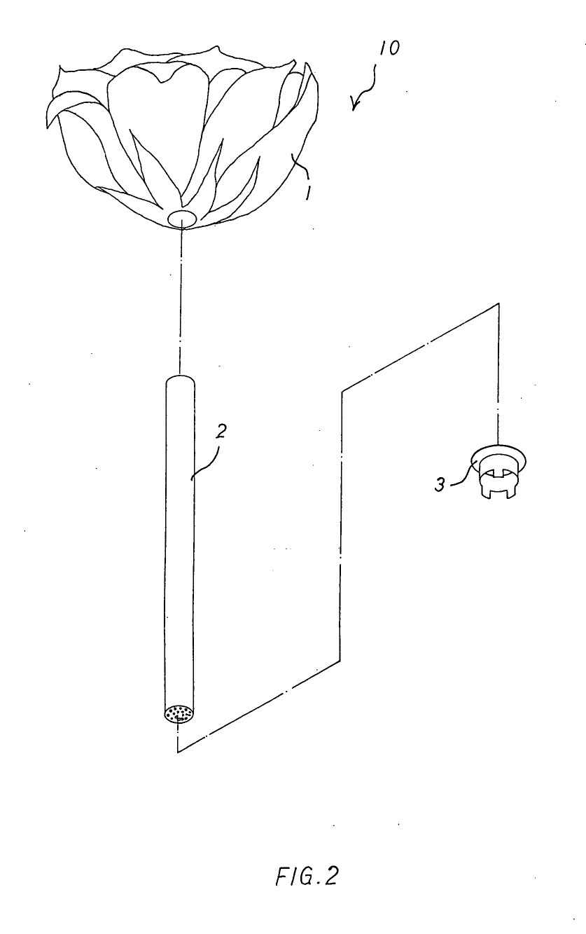

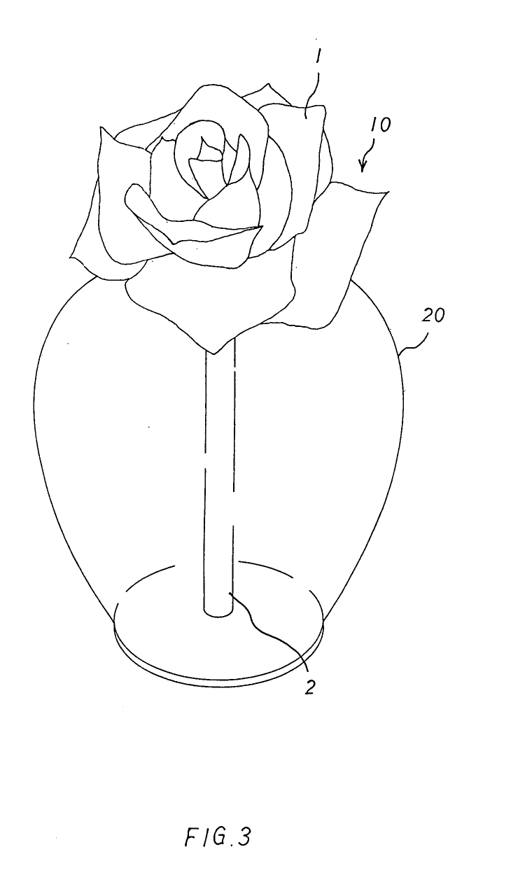

Referring to the drawings and initially to FIGS. 1 and 2, a core assembly 10 for essential oil in accordance with the preferred embodiment of the present invention comprises an oil suction member 2, a core head 1 mounted on and connected to a top end of the oil suction member 2, and a mounting seat 3 mounted between the oil suction member 2 and the core head 1.

The core head 1 is made of paper material and has a predetermined pattern in the form of a flower or the like. The core head 1 is bonded on the top end of the oil suction member 2 by waterproof adhesive. The mounting seat 3 is mounted on a connection between the oil suction member 2 and the core head 1 to combine the oil suction member 2 with the core head 1.

As shown in FIG. 3, when the core assembly 10 is mounted on a container 20, the oil suction member 2 is inserted into the container 20, the mounting seat 3 is mounted on a mouth of the container 20, and the core head 1 is protruded from the container 20. Thus, the core head 1 is combined with the oil suction member 2, so that after the oil suction member 2 absorbs the essential oil contained in the container 20, the essential oil is absorbed rapidly by the core head 1 by means of the capillary effect the oil suction member 2. At this time, the core head 1 is in contact with the ambient air so that the essential oil is volatilized to provide a fragrant smell.

In such a manner, the essential oil is volatilized naturally without 5 having to heat the core assembly 10, so that the core assembly 10 is used safely, thereby preventing the core assembly 10 from causing danger to a user due to a heating action.

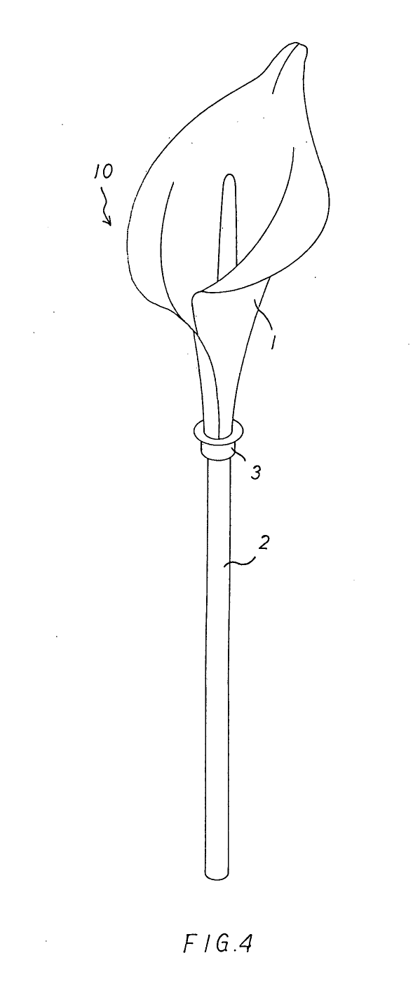

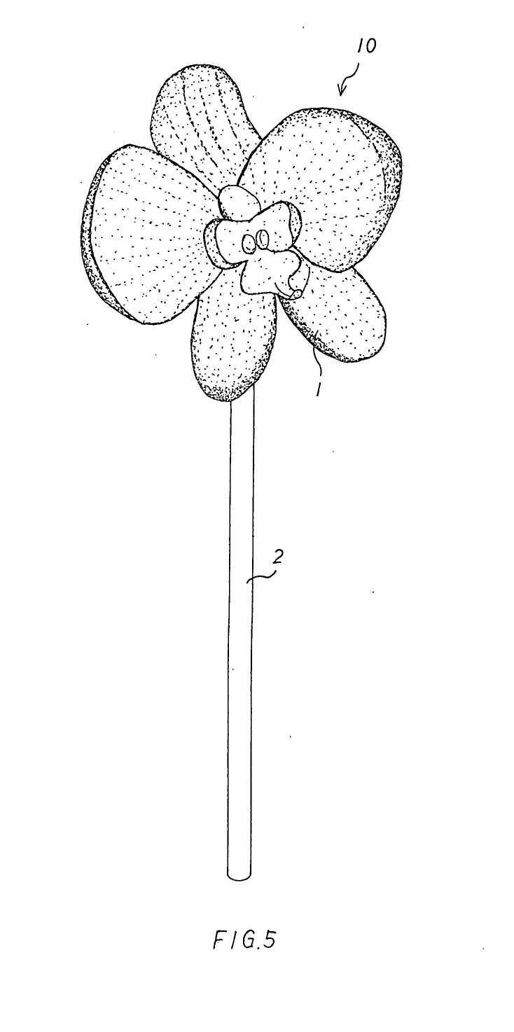

As shown in FIGS. 4 and 5, the core head 1 has different patterns.

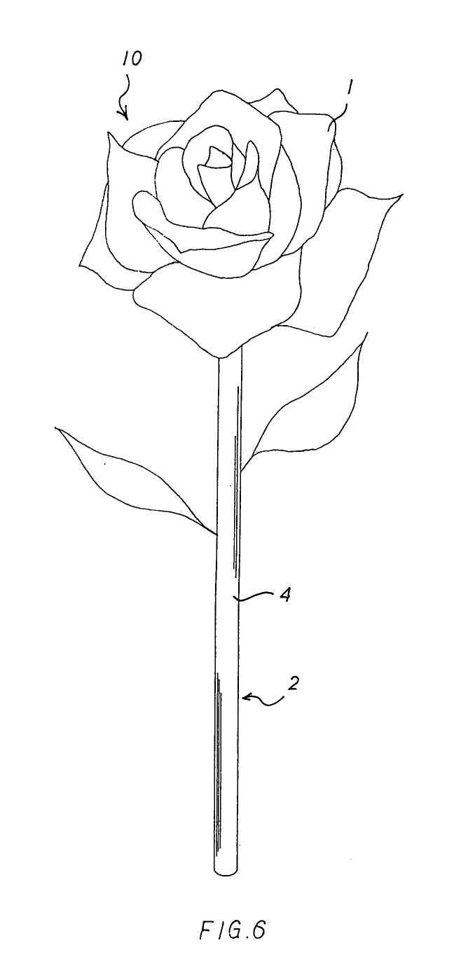

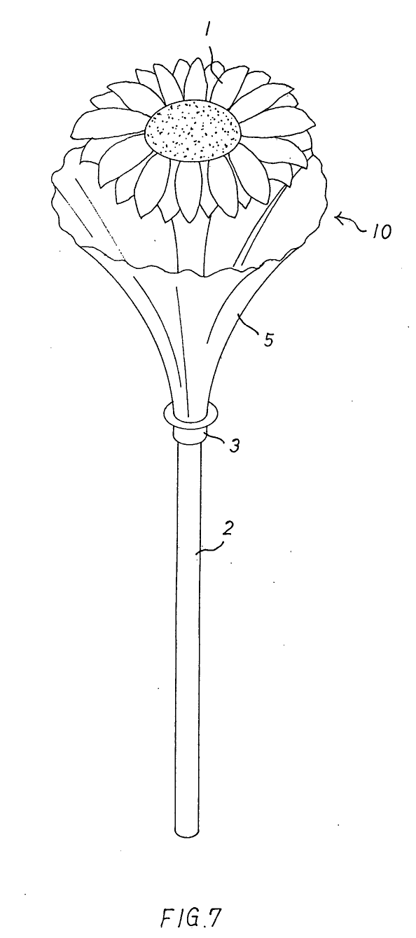

Referring to FIG. 6, after the oil suction member 2 absorbs saturated I 0 essential oil, a contraction film 4 is coated around a periphery of the oil suction member 2 to closely seal the oil suction member 2, so that the core assembly 10 is portable to facilitate storage and transportation of the core assembly 10.

Referring to FIG. 7, a funnel-shaped receiving seat 5 is mounted between the oil suction member 2 and the core head 1 and has an open area 15 greater than that of the core head 1 so as to receive the excessive essential oil dropped from the core head 1. In addition, the receiving seat 5 has a shape matching the pattern of the core head 1, thereby enhancing the aesthetic quality of the core assembly 10.

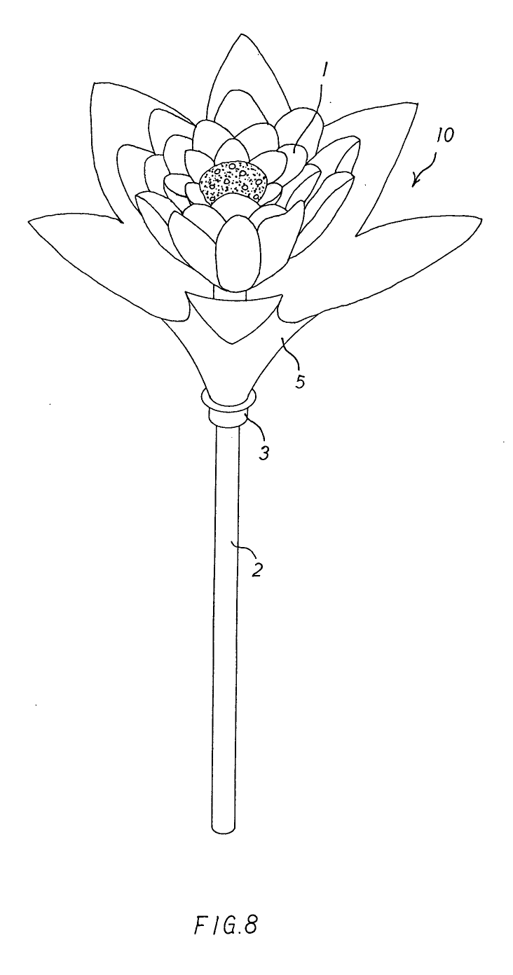

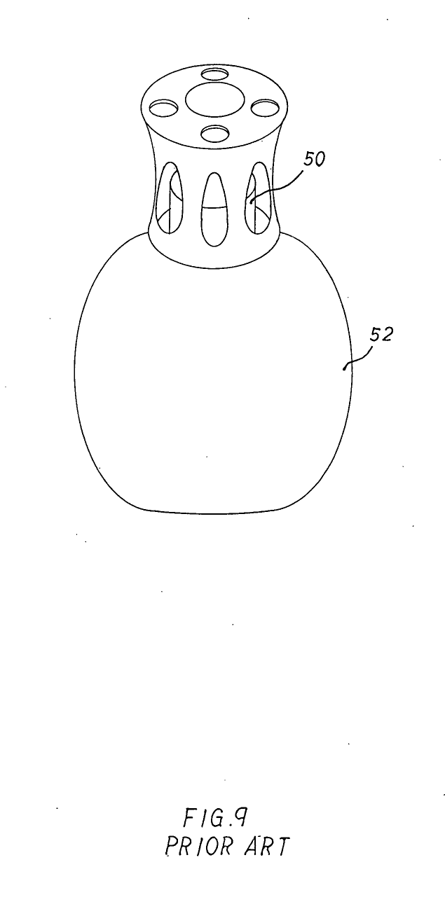

As shown in FIG. 8, the receiving seat 5 has a different shape. 20 Accordingly, the core head 1 is combined with the oil suction member 2, so that the essential oil absorbed by the capillary effect the oil suction member 2 is distributed on the core head 1 evenly and smoothly so as to contact with the air completely, thereby enhancing the volatile effect of the essential oil so as to provide a fragrant smell. In addition, the essential oil is volatilized naturally without having to heat the core assembly 10, so that the core assembly 10 is used safely, thereby preventing the core assembly 10 from causing danger to a user due to a heating action. Further, the core head 1 is made of paper material, thereby decreasing costs of fabrication. Further, the core head 1 has different patterns, thereby enhancing the aesthetic quality of the core assembly 10. Further, after the oil suction member 2 absorbs saturated essential oil, a contraction film 4 is coated around a periphery of the oil suction member 2 to closely seal the oil suction member 2, so that the core assembly 10 is portable to facilitate storage and transportation of the core assembly 10.

Although the invention has been explained in relation to its preferred embodiment(s) as mentioned above, it is to be understood that many other possible modifications and variations can be made without departing from the scope of the present invention. It is, therefore, contemplated that the appended claim or claims will cover such modifications and variations that fall within the true scope of the invention.

What is claimed is:

1. A core assembly, comprising:
an oil suction member;
a core head mounted on and connected to a top end of the oil suction member.

2. The core assembly in accordance with claim 1, further comprising a mounting seat mounted between the oil suction member and the core head.

3. The core assembly in accordance with claim 2, wherein the mounting seat is mounted on a connection between the oil suction member and the core head to combine the oil suction member with the core head.

4. The core assembly in accordance with claim 1, wherein the core head is made of paper material.

5. The core assembly in accordance with claim 1, wherein the core head has a predetermined pattern.

6. The core assembly in accordance with claim 1, wherein the core head is bonded on the top end of the oil suction member by waterproof adhesive.

7. The core assembly in accordance with claim 1, wherein the core head has different patterns.

8. The core assembly in accordance with claim 1, further comprising a contraction film coated around a periphery of the oil suction member to closely seal the oil suction member, so that the core assembly is portable to facilitate storage and transportation of the core assembly.

9. The core assembly in accordance with claim 8, wherein the contraction film is coated around the periphery of the oil suction member after the oil suction member absorbs saturated essential oil.

10. The core assembly in accordance with claim 1, further comprising a funnel-shaped receiving seat mounted between the oil suction member 2 and the core head.

11. The core assembly in accordance with claim 10, wherein the receiving seat has an open area greater than that of the core head so as to receive excessive essential oil dropped from the core head.

12. The core assembly in accordance with claim 10, wherein the receiving seat 5 has a shape matching the pattern of the core head.
